(19) United States
(12) Patent Application Publication
(10) Pub. No.: US 2002/0016342 A1
(43) Pub. Date: Feb. 7, 2002

(54) Combination therapy using COX-2 selective inhibitor and thromboxane inhibitor and compositions therefor

(76) Inventors: Edward Scolnick, Wynnewood, PA (US); Kathleen Metters, Montreal (CA); Denis Riendeau, Pierrefonds (CA); Mervyn Turner, Westfield, NJ (US)

Correspondence Address: MERCK AND CO INC, P O BOX 2000, RAHWAY, NJ, 070650907

(21) Appl. No.: 09/855,061

(22) Filed: May 14, 2001

Related U.S. Application Data
(60) Provisional application No. 60/204,269, filed on May 15, 2000.

(51) Int. Cl. A61K 31/444; A61K 31/427; A61K 31/365; A61K 31/122

(52) U.S. Cl. 514/332; 514/365; 514/473; 514/684

(57) ABSTRACT

The present invention provides a method for the treatment or prophylaxis of COX-2-mediated conditions in patients who are at risk of developing thromboembolic events which comprises administering to said patient a therapeutically or prophylactically effective amount of a COX-2 selective inhibitor and a cardiovascular protective amount of a thromboxane inhibitor, as well as compositions therefor.

### BACKGROUND OF THE INVENTION

Non-steroidal, antiinflammatory drugs exert most of their antiinflammatory, analgesic and antipyretic activity and inhibit hormone-induced uterine contractions and certain types of cancer growth through inhibition of prostaglandin G/H synthase, also known as cyclooxygenase. Initially, only one form of cyclooxygenase was known, this corresponding to cyclooxygenase-1 (COX-1) or the constitutive enzyme. More recently a second, inducible, form of cyclooxygenase, cyclooxygenase-2 (COX-2) has been identified. COX-2 is rapidly and readily inducible by a number of agents including mitogens, endotoxin, hormones, cytokines and growth factors.

COX-1 is responsible, in large part, for endogenous basal release of prostaglandins and hence is important in their physiological functions such as the maintenance of gastrointestinal integrity and renal blood flow. In contrast, COX-2 is mainly responsible for the pathological effects of prostaglandins where rapid induction of the enzyme would occur in response to such agents as inflammatory agents, hormones, growth factors, and cytokines. Most traditional NSAIDs inhibit both COX-1 and COX-2 isoforms of cyclooxygenase, and therefore their desirable antiinflammatory effect is often accompanied by undesirable gastrointestinal damaging effect. Selective inhibitors of COX-2 have similar antiinflammatory, antipyretic and analgesic properties to a conventional non-steroidal antiinflammatory drug but have a diminished ability to induce some of the mechanism-based side effects. COX-2 selective inhibitors currently on the market, rofecoxib and celecoxib, have been shown to have much lower incidence of gastrointestinal side effects than traditional NSAIDs (NSAIDs that have no or little selectivity for COX-2 over COX-1).

Traditional NSAIDs also affect platelet function by virtue of their COX-1 inhibitory activity. Inhibition of COX-1 prevents the formation in platelet of thromboxane A2, a mediator that promotes platelet aggregation. This effect of NSAIDs on platelet function has been exploited therapeutically, as in the case of aspirin, in the prophylaxis of thromboembolic disorders. COX-2 inhibitors, on the other hand, are not expected to have such protective effect.

In patients who are taking COX-2 selective inhibitors, those who are at risk of developing thromboembolic event may benefit from the anti-platelet aggregation effect of traditional NSAIDs, such as aspirin. However, the chronic use of aspirin for its cardiovascular protective effect, albeit at doses lower than normally used for its antiinflammatory effect, would undesirably expose these patients to gastrointestinal side effects while they are on an otherwise GI-sparing treatment regimen. Therefore, for patients who are taking COX-2 selective inhibitors and who may benefit from the cardiovascular protective effect of aspirin, there remains a need for a cardiovascular protective treatment that does not expose them to increased risk for gastrointestinal side effects.

PCT Published Application WO00/18352 discloses a method for treating inflammtory diseases by administering a thrombin inhibitor, which may be used in combination of an NSAID, such as COX-2 inhibitors.

PCT Published Application WO99/45913 discloses combination therapy and composition for acute coronary ischemic syndrome using an antiplatelet agent and a COX-2 inhibitor.

### SUMMARY OF THE INVENTION

The present invention concerns a method for treating patients with COX-2-mediated conditions, and who are also at risk of developing thromboembolic events which comprises administering to said patients a COX-2 selective inhibitor and a thromboxane A2 inhibitor. Also provided are pharmaceutical compositions comprising a COX-2 selective inhibitor and a thromboxane inhibitor.

### DETAILED DESCRIPTION OF THE INVENTION

The present invention provides a novel method for the treatment or prophylaxis of COX-2-mediated conditions in patients who are at risk of developing thromboembolic events which comprises administering to said patients a therapeutically or prophylactically effective amount of a COX-2 selective inhibitor and a cardiovascular protective amount of a thromboxane inhibitor.

The present invention also provides for pharmaceutical compositions comprising a therapeutically or prophylactically effective amount of a COX-2 selective inhibitor and a cardiovascular protective amount of a thromboxane inhibitor, and a pharmaceutically acceptable carrier in unit dosage form.

The present invention also provides a pharmaceutical product comprising (1) a therapeutically or prophylactically effective amount of a COX-2 selective inhibitor in a first oral unit dosage form, (2) a cardioprotective amount of a thromboxane inhibitor in a second oral unit dosage form, and (3) instructions for concurrent or sequential administration of said pharmaceutical product to a patient in need thereof.

In one embodiment of the present method, the COX-2 mediated condition is selected from osteoarthritis, rheumatoid arthritis, cancer and Alzheimer's disease. In one subset the COX-2 mediated condition is cancer. In another subset the COX-2 mediated condition is Alzheimer's disease; in yet another subset, the COX-2 mediated condition is osteoarthritis or rheumatoid arthritis.

In another embodiment the COX-2 selective inhibitor and the thromboxane inhibitor are administered orally.

In another embodiment the COX-2 selective inhibitor is selected from celecoxib, rofecoxib, valdecoxib and etoricoxib.

“Conditions mediated by COX-2” include, but are not limited to, pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, injuries following surgical and dental procedures, cancer including the transformation of a colonic adenoma to a colonic adenocarcinoma, and dementia including pre-senile and senile dementia, and in particular, dementia associated with Alzheimer Disease (ie Alzheimer's dementia).

“Thromboembolic event” includes, but are not limited to, ischemic stroke, transient ischemic stroke, and myocardial infarction. “Patients who are at risk of developing thromboembolic events” include those with a familial history of, or genetically predisposed to, thromboembolic disorders, who have had ischemic stroke, transient ischemic stroke, myocardial infarction, and those with unstable angina pectoris or chronic stable angina pectoris and patients with altered prostacyclin/thromboxane A₂ homeostasis or higher than normal thromboxane A₂ levels leading to increase risk for thromboembolism, including patients with diabetes and rheumatoid arthritis.

“COX-2 selective inhibitors” embrace compounds which selectively inhibit cyclooxygenase-2 over cyclooxygenase-1. COX-2 and COX-1 inhibitory activities may be determined employing the human whole blood COX-1 assay and the human whole blood COX-2 assay described in C. Brideau et al, Inflamm. Res. 45: 68-74 (1996), herein incorporated by reference. Preferably, the compounds have a cyclooxygenase-2 IC₅₀ of less than about 2 μM in the human whole blood COX-2 assay, yet have a cyclooxygenase-1 IC₅₀ of greater than about 5 μM in the human whole blood COX-1 assay. Also preferably, the compounds have a selectivity ratio of cyclooxygenase-2 inhibition over cyclooxygenase-1 inhibition of at least 5, and more preferably of at least 30.

“Thromboxane inhibitors” include compounds that inhibit thromboxane synthase and compounds that inhibit, prevent or otherwise interfere with the binding of thromboxane to its receptor (thromboxane antagonists), as well as compounds that are both thromboxane synthase inhibitors and thromboxane receptor antagonists. Thromboxane synthase inhibitors and thromboxane receptor antagonists can be identified using assays described in Tai, H. -H. Assay of thromboxane A synthase inhibitors. Methods in Enzymology Vol 86, 1982 pp. 110-113 and references contained within Hall, S. E. Thromboxane A₂ Receptor Antagonists. Medicinal Research Reviews, 11, 503-579 (1991) and Coleman, R. A., Smith, W. L., Narumiya, S. International Union of Pharmacology classification of prostanoid receptors: properties, distribution and structure of the receptors and their subtypes. Pharmacol. Rev. 46, 205-229 (1994). The characteristics of the preferred thromboxane inhibitor should include suppression of thromboxane A₂ formation (thromboxane synthase inhibitors) and/or blockade of thromboxane A₂ and prostaglandin H₂ on platelets and vessel wall (thromboxane receptor antagonists). The effects should block platelet activation and therefore platelet function. Thromboxane synthase inhibitors may also increase the synthesis of antiaggregatory prostaglandins including prostacyclin and prostaglandin D₂.

“Therapeutically effective amount” means that amount of a drug or pharmaceutical agent that will elicit the biological or medical response of a tissue, a system, animal or human that is being sought by a researcher, veterinarian, medical doctor or other clinician.

The term “treatment” or “treating” includes alleviating, ameliorating, relieving or otherwise reducing the signs and symptoms associated with a disease or disorder.

The term “prophylaxis” means preventing or delaying the onset or the progression of a disease or disorder, or the signs and symptoms associated with such disease or disorder.

“Prophylactically effective amount” means that amount of a pharmaceutical drug that will prevent, delay or reduce the risk of occurrence of the biological or medical event that is sought to be prevented in a tissue, a system, animal or human by a researcher, veterinarian, medical doctor or other clinician.

“Cardiovascular protective amount” means that amount of a thromboxane inhibitor that will prevent or reduce the risk of occurrence of thromboembolic events.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a COX-2 selective inhibitor and a thromboxane inhibitor, and pharmaceutically acceptable excipients.

COX-2 Selective Inhibitors

As explained in J. Talley, Exp. Opin. Ther. Patents (1997), 7(1), pp. 55-62, three distinct structural classes of selective COX-2 inhibitor compounds have been identified. One class is the methane sulfonanilide class of inhibitors, of which NS-398, flosulide, nimesulide and (i) are example members.

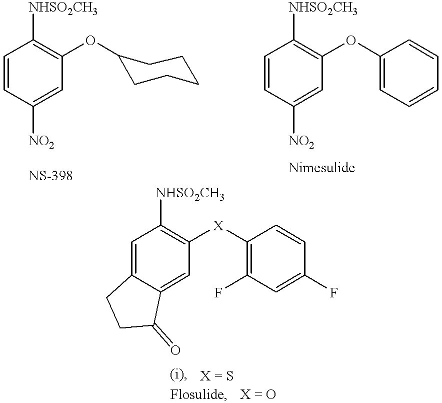

A second class is the tricyclic inhibitor class, which can be further divided into the sub-classes of tricyclic inhibitors with a central carbocyclic ring (examples include SC-57666, 1, and 2); those with a central monocyclic heterocyclic ring (examples include DuP 697, SC-58125, SC-58635, and 3, 4 and 5; and those with a central bicyclic heterocyclic ring (examples include 6, 7, 8, 9 and 10). Compounds 3, 4 and 5 are described in U.S. Pat. No. 5,474,995.

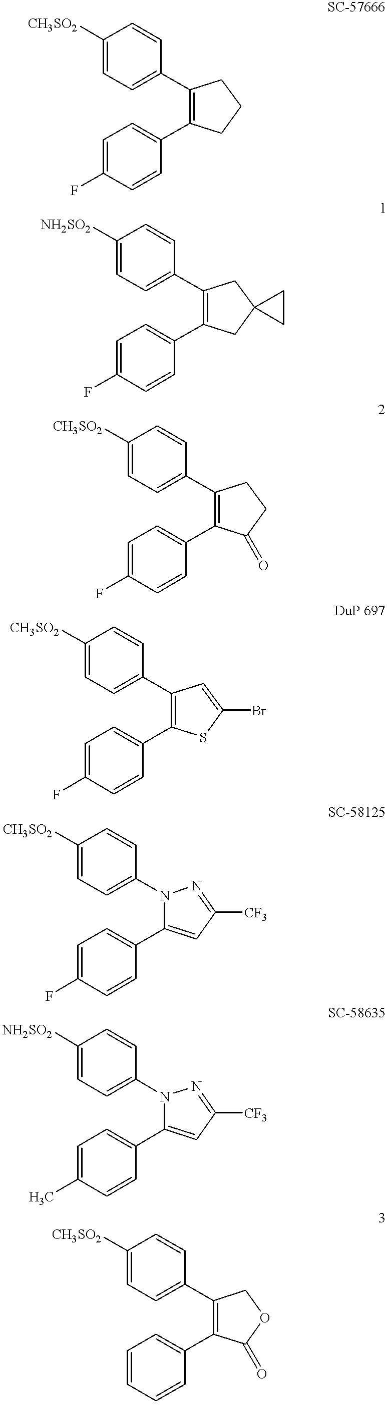

The third identified class can be referred to as those which are structurally modified NSAIDs, and includes 11a and structure 11 as example members.

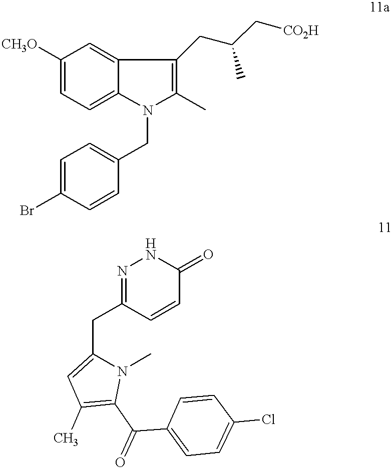

In addition to the structural classes, sub-classes, specific COX-2 selective inhibitor compound examples, and reference journal and patent publications described in the Talley publication which are all herein incorporated by reference, examples of compounds which selectively inhibit cyclooxygenase-2 have also been described in the following patent publications, all of which are herein incorporated by reference: U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, 5,639,780; and International Patent Specification Nos. 94/13635, 94/15932, 94/20480, 94/26731, 94/27980, 95/00501, 95/15316, 96/03387, 96/03388, 96/06840; and International Publication Nos. WO 94/20480, WO 96/21667, WO 96/31509, WO 96/36623, WO 97/14691, WO 97/16435.

Additional COX-2 selective inhibitor compounds which are included in the scope of this invention include:

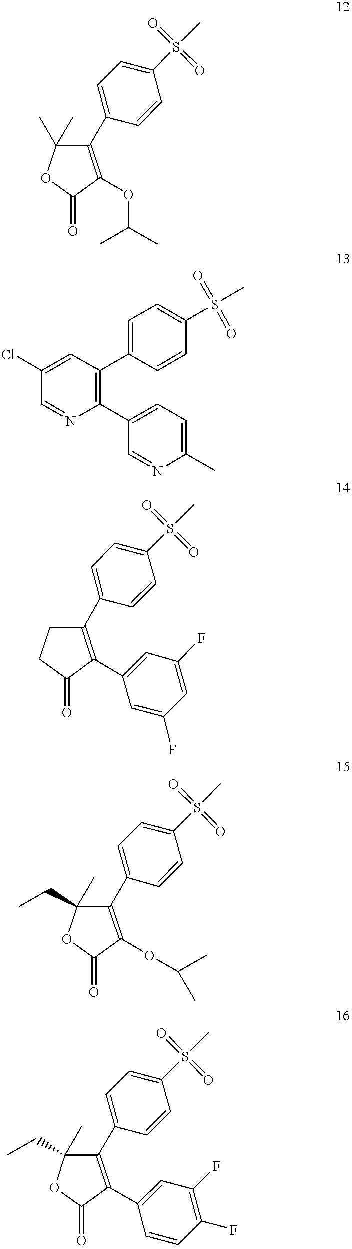

Some of the compounds above can also be identified by the following chemical names:

SC58635: 4-[5-(4-methylphenyl)-3-(trifluoromethyl)-1H-pyrazol-1-yl]benzene-sulfonamide (celecoxib);

3: 3-phenyl-4-(4-(methylsulfonyl)phenyl)-2-(5H)-furanone (rofecoxib);

4: 3-(3,4-difluorophenyl)-4-(4-(methylsulfonyl)phenyl)-2-(5H)-furanone;

5: 5,5-dimethyl-4-(4-(methylsulfonyl)phenyl)-3-(3-fluorophenyl)-5H-furan-2-one;

12: 5,5-dimethyl-4-(4-(methylsulfonyl)phenyl)-3-(2-propoxy)-5H-furan-2-one

13: 5-chloro-3-(4-(methylsulfonyl)phenyl)-2-(2-methyl-5-pyridinyl)pyidine (etoricoxib);

14: 2-(3,5-difluorophenyl)-3-(4-(methylsulfonyl)phenyl)-2-cyclopenten-1-one

15: 5(S)-ethyl-5-methyl-4-(4-(methylsulfonyl)phenyl)-3-(2-propoxy)-5H-furan-2-one

16: 5-ethyl-5-methyl-4-(4-(methylsulfonyl)phenyl)-3-(3,4-difluorophenyl)-5H-furan-2-one;

17: 3-((2-thiazolyl)methoxy)-4-(4-(methylsulfonyl)phenyl)-5,5-dimethyl-5H-furan-2-one

18: 3-propyloxy-4-(4-(methylsulfonyl)phenyl)-5,5-dimethyl-5H-furan-2-one

19: 3-(1-cyclopropylethoxy)-5,5-dimethyl-4-(4-methylsulfonyl)phenyl)-5H-furan-2-one

20: sodium 2-(4-chlorophenyl)-3-(4-(methylsulfonyl)phenyl)-4-oxo-2-pentenoate;

21: 3-(cyclopropylmethoxy)-5,5-dimethyl-4-(4-(methylsulfonyl)phenyl)-SH-furan-2-one

22: 3-(cyclopropylmethoxy)-5,5-dimethyl-4-(4-(methylsulfonyl)phenyl)-2,5-dihydro-furan-2-ol;

23: 3-isopropoxy-5,5-dimethyl-4-(4-(methylsulfonyl)phenyl)-2,5-dihydrofuran-2-ol

24: 5,5-dimethyl-3-(3-fluorophenyl)-2-hydroxy-4-(4-(methylsulfonyl)phenyl)-2,5-dihydrofuran

25: 5-Chloro-3-(4-(methylsulfonyl)phenyl)-2-(3-pyridinyl)pyridine

The following publications describe and/or provide methods for making the compounds as indicated: compounds 12, 15, 17, 18, 19 and 21, WO 97/14691; compounds 22, 23 and 24, WO 97/16435; compound 20, WO 96/36623; compound 14, U.S. Pat. No. 5,536,752; compound 16, U.S. Pat. No. 5,474,995. See Examples herein for compounds 13 and 25.

Also incorporated herein by reference are those compounds described in WO 96/41645 as having structural Formula I, shown below, and the definition and preferred definitions and species described therein:

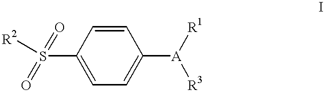

Particularly preferred compounds of formula (I) include:

5-(4-fluorophenyl)-1-[4-(methylsulfonyl)phenyl]-3-(trifluoromethyl)pyrazole;

4-(4-fluorophenyl)-5-[4-(methylsulfonyl)phenyl]-1-phenyl-3-(trifluoromethyl)-pyrazole;

4-(5-(4-chlorophenyl)-3-(4-methoxyphenyl)-1H-pyrazol-1-yl)benzenesulfonamide;

4-(3,5-bis(4-methylphenyl)-1H-pyrazol-1-yl)benzenesulfonamide;

4-(5-(4-chlorophenyl)-3-phenyl-1H-pyrazol-1-yl)benzenesulfonamide;

4-(3,5-bis(4-methoxyphenyl)-1H-pyrazol-1-yl)benzenesulfonamide;

4-(5-(4-chlorophenyl)-3-(4-methylphenyl)-1H-pyrazol-1-yl)benzenesulfonamide;

4-(5-(4-chlorophenyl)-3-(4-nitrophenyl)-1H-pyrazol-1-yl)benzenesulfonamide;

4-(5-(4-chlorophenyl)-3-(5-chloro-2-thienyl)-1H-pyrazol-1-yl)benzenesulfonamide;

4-(4-chloro-3,5-diphenyl-1H-pyrazol-1-yl)benzenesulfonamide;

4-(5-(4-chlorophenyl)-3-(trifluoromethyl)-1H-pyrazol-1-yl)benzenesulfonamide;

4-(5-phenyl)-3-(trifluoromethyl)-1H-pyrazol-1-yl)benzenesulfonamide;

4-(5-(4-fluorophenyl)-3-(trifluoromethyl)-1H-pyrazol-1-yl)benzenesulfonamide;

4-(5-(4-methoxyphenyl)-3-(trifluoromethyl)-1H-pyrazol-1-yl)benzenesulfonamide;

4-(5-(4-chlorophenyl)-3-(difluoromethyl)-1H-pyrazol-1-yl)benzenesulfonamide;

4-(5-(4-methylphenyl)-3-(trifluoromethyl)-1H-pyrazol-1-yl)benzenesulfonamide;

4-(4-chloro-5-(4-chlorophenyl)-3-(trifluoromethyl)-1H-pyrazol-1-yl)benzenesulfonamide;

4-(3-(difluoromethyl)-5-(4-methylphenyl)-1H-pyrazol-1-yl)benzenesulfonamide;

4-(3-(difluoromethyl)-5-phenyl-1-H-pyrazol-1-yl) benzenesulfonamide;

4-(3-(difluoromethyl)-5-(4-methoxyphenyl)-1H-pyrazol-1-yl)benzenesulfonamide;

4-(3-cyano-5-(4-fluorophenyl)-1H-pyrazol-1-yl)benzenesulfonamide;

4-(3-(difluoromethyl)-5-(3-fluoro-4-methoxyphenyl)-1H-pyrazol-1-yl)benzenesulfonamide;

4-(5-(3-fluoro-4-methoxyphenyl)-3-(trifluoromethyl)-1H-pyrazol-1-yl)benzenesulfonamide;

4-(4-chloro-5-phenyl-1H-pyrazol-1-yl)benzenesulfonamide;

4-(5-(4-chlorophenyl)-3-(hydroxyphenyl)-1H-pyrazol-1-yl)benzenesulfonamide;

4-(5-(4-(N,N-dimethylamino)phenyl)-3-(trifluoromethyl)-1H-pyrazol-1-yl)benzenesulfonamide;

5-(4-fluorophenyl)-6-(4-(methylsulfonyl)phenyl)spiro [2.4]hept-5-ene;

4-(6-(4-fluorophenyl)spiro[2.4]hept-5-en-5-yl)benzenesulfonamide;

6-(4-fluorophenyl)-7-(4-(methylsulfonyl)phenyl)spiro[3.4]oct-6-ene;

5-(3-chloro-4-methoxyphenyl)-6-(4-(methylsulfonyl)phenyl)spiro[2.4]hept-5-ene;

4-(6-(3-chloro-4-methoxyphenyl)spiro[2.4]hept-5-en-5-yl)benzenesulfonamide;

5-(3,5-dichloro-4-methoxyphenyl)-6-(4-(methylsulfonyl)phenyl)spiro [2.4]hept-5-ene;

5-(3-chloro-4-fluorophenyl)-6-(4-(methylsulfonyl)phenyl)spiro[2.4]hept-5-ene;

4-(6-(3,4-dichlorophenyl)spiro[2.4]hept-5-en-5-yl)benzenesulfonamide;

2-(3-chloro-4-fluorophenyl)-4-(4-fluorophenyl)-5-(4-methylsulfonylphenyl)thiazole;

2-(2-chlorophenyl)-4-(4-fluorophenyl)-5-(4-methylsulfonylphenyl)thiazole;

5-(4-fluorophenyl)-4-(4-methylsulfonylphenyl)-2-methylthiazole;

4-(4-fluorophenyl)-5-(4-methylsulfonylphenyl)-2-trifluoromethylthiazole;

4-(4-fluorophenyl)-5-(4-methylsulfonylphenyl)-2-(2-thienyl)thiazole;

4-(4-fluorophenyl)-5-(4-methylsulfonylphenyl)-2-benzylaminothiazole;

4-(4-fluorophenyl)-5-(4-methylsulfonylphenyl)-2-(1-propylamino)thiazole;

2-((3,5-dichlorophenoxy)methyl)-4-(4-fluorophenyl)-5-(4-(methylsulfonyl)-phenyl)thiazole;

5-(4-fluorophenyl)-4-(4-methylsulfonylphenyl)-2-trifluoromethylthiazole;

1-methylsulfonyl-4-(1,1-dimethyl-4-(4-fluorophenyl)cyclopenta-2,4-dien-3-yl)-benzene;

4-(4-(4-fluorophenyl)-1,1-dimethylcyclopenta-2,4-dien-3-yl)benzenesulfonamide;

5-(4-fluorophenyl)-6-(4-(methylsulfonyl)phenyl)spiro [2.4]hepta-4,6-diene;

4-(6-(4-fluorophenyl)spiro[2.4]hepta-4,6-dien-5-yl)benzenesulfonamide;

6-(4-fluorophenyl)-2-methoxy-5-(4-(methylsulfonyl)phenyl)-pyridine-3-carbonitrile;

2-bromo-6-(4-fluorophenyl)-5-(4-(methylsulfonyl)phenyl)-pyridine-3-carbonitrile;

4-(2-(4-methylpyridin-2-yl-4-(trifluoromethyl)-1H-imidazol-1-yl)benzenesulfonamide;

4-(2-(5-methylpyridin-3-yl)-4-(trifluoromethyl)-1H-imidazol-1-yl)benzenesulfonamide;

4-(2-(2-methylpyridin-3-yl)-4-(trifluoromethyl)-1H-imidazol-1-yl)benzenesulfonamide;

3-(1-(4-(methylsulfonyl)phenyl)-4-(trifluoromethyl)-1H-imidazol -2-yl)benzenesulfonamide;

2-(1-(4-(methylsulfonyl)phenyl)-4-(trifluoromethyl)-1H-imidazol-2-yl)pyridine;

2-methyl-4-(1-(4-(methylsulfonyl)phenyl)-4-(trifluoromethyl)-1H-imidazol-2-yl)-pyridine;

2-methyl-6-(1-(4-(methylsulfonyl)phenyl)-4-(trifluoromethyl)-1H-imidazol-2-yl)-pyridine;

4-(2-(6-methylpyridin-3-yl)-4-(trifluoromethyl)-1H-imidazol-1-yl)benzenesulfonamide;

2-(3,4-difluorophenyl)-1-(4-(methylsulfonyl)phenyl)-4-(trifluoromethyl)-1H-imidazole;

4-(2-(4-methylphenyl)-4-(trifluoromethyl)-1H-imidazol-1-yl)benzenesulfonamide;

2-(4-chlorophenyl)-1-(4-(methylsulfonyl)phenyl)-4-methyl-1H-imidazole;

2-(4-chlorophenyl)-1-(4-(methylsulfonyl)phenyl)-4-phenyl-1H-imidazole;

2-(4-chlorophenyl)-4-(4-fluorophenyl)-1-(4-(methylsulfonyl)phenyl)-1H-imidazole;

2-(3-fluoro-4-methoxyphenyl)-1-(4-(methylsulfonyl)phenyl)-4-(trifluoromethyl)-1H-imidazole;

1-(4-(methylsulfonyl)phenyl)-2-phenyl-4-trifluoromethyl-1H-imidazole;

2-(4-methylphenyl)-1-(4-(methylsulfonyl)phenyl)-4-trifluoromethyl-1H-imidazole;

4-(2-(3-chloro-4-methylphenyl)-4-(trifluoromethyl)-1H-imidazol-1-yl)benzenesulfonamide;

2-(3-fluoro-5-methylphenyl)-1-(4-(methylsulfonyl)phenyl)-4-(trifluoromethyl)-1H-imidazole;

4-(2-(3-fluoro-5-methylphenyl)-4-(trifluoromethyl)-1H-imidazol-1-yl)benzenesulfonamide;

2-(3-methylphenyl)-1-(4-(methylsulfonyl)phenyl)-4-(trifluoromethyl)-1H-imidazole;

4-(2-(3-methylphenyl)-4-(trifluoromethyl)-1H-imidazol-1-yl)benzenesulfonamide;

1-(4-(methylsulfonyl)phenyl)-2-(3-chlorophenyl)-4-(trifluoromethyl)-1H-imidazole;

4-(2-(3-chlorophenyl)-4-(trifluoromethyl)-1H-imidazol-1-yl)benzenesulfonamide;

4-(2-phenyl-4-(trifluoromethyl)-1H-imidazol-1-yl) benzenesulfonamide;

4-(2-(4-methoxy-3-chlorophenyl)-4-(trifluoromethyl)-1H-imidazol-1-yl)benzenesulfonamide;

1-allyl-4-(4-fluorophenyl)-3-(4-(methylsulfonyl)phenyl)-5-(trifluoromethyl)-1H-pyrazole;

4-(1-ethyl-4-(4-fluorophenyl)-5-(trifluoromethyl)-1H-pyrazol-3-yl)benzenesulfonamide;

N-phenyl-(4-(4-fluorophenyl)-3-(4-(methylsulfonyl)phenyl)-5-(trifluoromethyl)-1H-pyrazol-1-yl)acetamide;

ethyl (4-(4-fluorophenyl)-3-(4-(methylsulfonyl)phenyl)-5-(trifluoromethyl)-1H-pyrazol-1-yl)acetate;

4-(4-fluorophenyl)-3-(4-(methylsulfonyl)phenyl)-1-(2-phenylethyl)-1H-pyrazole;

4-(4-fluorophenyl)-3-(4-(methylsulfonyl)phenyl)-1-(2-phenylethyl)-5-(trifluoromethyl)pyrazole;

1-ethyl-4-(4-fluorophenyl)-3-(4-(methylsulfonyl)phenyl)-5-(trifluoromethyl)-1H-pyrazole;

5-(4-fluorophenyl)-4-(4-(methylsulfonyl)phenyl)-2-(trifluoromethyl)-1H-imidazole;

4-(4-(methylsulfonyl)phenyl)-5-(2-thiophenyl)-2-(trifluoromethyl)-1H-imidazole;

5-(4-fluorophenyl)-2-methoxy-4-(4-(methylsulfonyl)phenyl)-6-(trifluoromethyl)-pyridine;

2-ethoxy-5-(4-fluorophenyl)-4-(4-(methylsulfonyl)phenyl)-6-(trifluoromethyl)-pyridine;

5-(4-fluorophenyl)-4-(4-(methylsulfonyl)phenyl)-2-(2-propynyloxy)-6-(trifluoromethyl)pyridine;

2-bromo-5-(4-fluorophenyl)-4-(4-(methylsulfonyl)phenyl)-6-(trifluoromethyl)-pyridine;

4-(2-(3-chloro-4-methoxyphenyl)-4,5-difluorophenyl)benzenesulfonamide;

1-(4-fluorophenyl)-2-(4-(methylsulfonyl)phenyl)benzene;

5-difluoromethyl-4-(4-(methylsulfonyl)phenyl)-3-phenylisoxazole;

4-(3-ethyl-5-phenylisoxazol-4-yl)benzenesulfonamide;

4-(5-difluoromethyl-3-phenylisoxazol-4-yl)benzenesulfonamide;

4-(5-hydroxymethyl-3-phenylisoxazol-4-yl)benzenesulfonamide;

4-(5-methyl-3-phenylisoxazol-4-yl)benzenesulfonamide (valdecoxib);

N-propanoyl-4-(5-methyl-3-phenylisoxazol-4-yl)benzenesulfonamide;

1-(2-(4-fluorophenyl)cyclopenten-1-yl)-4-(methylsulfonyl)benzenee;

1-(2-(4-fluoro-2-methylphenyl)cyclopenten-1-yl)-4-(methylsulfonyl)benzene;

1-(2-(4-chloro phenyl)cyclopenten-1-yl)-4-(methylsulfonyl)benzene;

1-(2-(2,4-dichlorophenyl)cyclopenten-1-yl)-4-(methylsulfonyl)benzene;

1-(2-(4-trufluoromethylphenyl)cyclopenten-1-yl)-4-(methylsulfonyl)benzene;

1-(2-(4-methylthiophenyl)cyclopenten-1-yl)-4-(methylsulfonyl)benzene;

1-(2-(4-fluorophenyl)-4,4-dimethylcyclopenten-1-yl)-4-(methylsulfonyl)benzene;

4-(2-(4-fluorophenyl)-4,4-dimethylcyclopenten-1-y l)benzenesulfonamide;

1-(2-(4-chlorophenyl)-4,4-dimethylcyclopenten-1-yl)-4-(methylsulfonyl)benzene;

4-(2-(4-chlorophenyl)-4,4-dimethylcyclopenten-1-yl)benzenesulfonamide;

4-(2-(4-fluorophenyl)cyclopenten-1-yl)benzenesulfonamide;

4-(2-(4-chlorophenyl)cyclopenten-1-yl)benzenesulfonamide;

1-(2-(4-methoxyphenyl)cyclopenten-1-yl)-4-(methylsulfonyl)benzene;

1-(2-(2,3-difluorophenyl)cyclopenten-1-yl)-4-(methylsulfonyl)benzene;

4-(2-(3-fluoro-4-methoxyphenyl)cyclopenten-1-yl)benzenesulfonamide;

1-(2-(3-chloro-4-methoxyphenyl)cyclopenten-1-yl)-4-(methylsulfonyl)benzene;

4-(2-(3-chloro-4-fluorophenyl)cyclopenten-1-yl)benzenesulfonamide;

4-(2-(2-methylpyridin-5-yl)cyclopenten-1-yl)benzenesulfonamide;

ethyl 2-(4-(4-fluorophenyl)-5-(4-(methylsulfonyl)phenyl)oxazol-2-yl)-2-benzyl-acetate;

2-(4-(4-fluorophenyl)-5-(4-(methylsulfonyl)phenyl)oxazol-2-yl)acetic acid;

2-(tert-butyl)-4-(4-fluorophenyl)-5-(4-(methylsulfonyl)phenyl)oxazole;

4-(4-fluorophenyl)-5-(4-(methylsulfonyl)phenyl)-2-phenyloxazole;

4-(4-fluorophenyl)-2-methyl-5-(4-(methylsulfonyl)phenyl)oxazole; and

4-(5-(3-fluoro-4-methoxyphenyl)-2-trifluoromethyl-4-oxazolyl)benzenesulfonamide; or a pharmaceutically acceptable salt thereof.

Several of the above mentioned COX-2 selective inhibitors have been approved for human use or are in advanced stage of development; accordingly, one subset of COX-2 selective inhibitors of the present invention include celecoxib, rofecoxib, valdecoxib and etoricoxib.

Thromboxane Inhibitors

Examples of thromboxane inhibitors include serabenast (seratrodast), picotamide, ozagrel, egualen, domitroban, ramatroban, ridrogrel, samixogrel, terbogrel, satrigrel, sulotraban, ifetroban, vapiprost, daltroban, imitrodast, dazoxiben, linotroban, triletide, nafagrel, rolafagrel, pirmagrel,

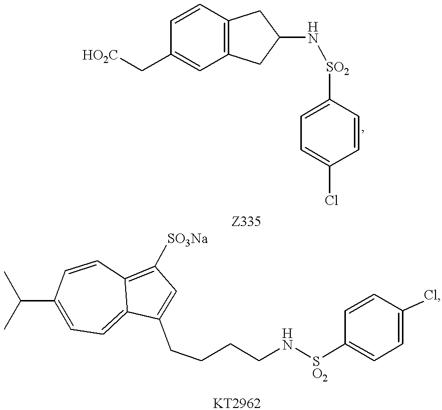

S18886, S32080, ICI192605, ICI185282, ONO-3708, ONO-8809, FPL-55712, WHR-2348, KW3635, LCB2853, Y20811, CGS12970, CGS22652, UK34787, MED27, ONO1301, MR948, AZ1355, KD1792 and F10171. Examples of thromoboxane inhibitors such as (−)-6,8-difluoro-9-p-methylsulfonylbenzyl-1,2,3,4-tetrahydrocarbazol-1-yl-acetic acid may also be found in U.S. Pat. No. 4,808,608, which is hereby incorporated by reference.

As used herein “COX-2 selective inhibitors” and “thromboxane inhibitors” (including thromboxane synthase inhibitors and thromboxane receptor antagonists) encompass pharmaceutically acceptable salts of the active chemical entity.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When a compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

Dosage and Administration

In the present method, the COX-2 selective inhibitor and the thromboxane inhibitor may be administered separately in separate dosage forms or together in a single unit dosage form. Where separate dosage formulations are used, the thromboxane inhibitor and the COX-2 selective inhibitor can be administered at essentially the same time, i.e., concurrently, or at separately staggered times, i.e, sequentially, and in any order. It is preferred that the thromboxane inhibitor and the COX-2 selective inhibitor be co-administered concurrently on a once-a-day dosing schedule; however, varying dosing schedules, such as the thromboxane inhibitor once per day and the COX-2 selective inhibitor once, twice or more times per day, or the COX-2 selective inhibitor once per day and the thromboxane inhibitor once, twice or more times per day, is also encompassed herein. A single oral dosage formulation comprised of both the thromboxane inhibitor and the COX-2 selective inhibitor is preferred. A single dosage formulation will provide convenience for the patient.

The COX-2 selective inhibitor may be administered at a dosage level up to conventional dosage levels for NSAIDs. Suitable dosage levels will depend upon the anti-inflammatory effect of the chosen inhibitor of cyclooxygenase-2, but typically suitable levels will be about 0.001 to 50 mg/kg body weight of the patient per day, preferably 0.005 to 30 mg/kg per day, and especially 0.05 to 10 mg/kg per day. The compound may be administered on a regimen of up to 6 times per day, preferably 1 to 4 times per day, and especially once per day.

In the case where an oral composition is employed, a suitable dosage range is, e.g. from about 0.01 mg to about 100 mg of a COX-2 selective inhibitor per kg of body weight per day, preferably from about 0.1 mg to about 10 mg per kg of a COX-2 selective inhibitor per kg of body weight per day.

The thromboxane inhibitor may be administered at a dosage level up to conventional dosage levels for thromboxane inhibitors. Suitable dosage levels will depend upon the cardiovascular protective effect of the chosen thromboxane inhibitor, but typically suitable levels will be about 0.001 to 50 mg/kg body weight of the patient per day, preferably 0.005 to 30 mg/kg per day, and especially 0.05 to 10 mg/kg per day. The compound may be administered on a regimen of up to 6 times per day, preferably 1 to 4 times per day, and especially once per day.

In the case where an oral composition is employed, a suitable dosage range is, e.g. from about 0.01 mg to about 100 mg of a thromboxane inhibitor per kg of body weight per day, preferably from about 0.1 mg to about 10 mg per kg and for cytoprotective use from 0.1 mg to about 100 mg of a thromboxane inhibitor per kg of body weight per day.

It will be understood that the dosage of the therapeutic agents will vary with the nature and the severity of the condition to be treated, and with the particular therapeutic agents chosen. The dosage will also vary according to the age, weight, physical condition and response of the individual patient. The selection of the appropriate dosage for the individual patient is within the skills of a clinician.

Pharmaceutical Compositions

Any suitable route of administration may be employed for providing a patient with an effective dosage of drugs of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. However, for the convenience of dosing, the drugs of the present invention are preferably administered orally.

The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the drugs used in the present invention are conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, drugs used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

The instant invention also provides pharmaceutical compositions comprised of a therapeutically effective amount of an COX-2 selective inhibitor in combination with a cardiovascular protective amount of a thromboxane inhibitor, and a pharmaceutically acceptable carrier. One embodiment of the instant compositions is a single unit dosage form adapted for oral administration comprised of a therapeutically effective amount of a COX-2 selective inhibitor in combination with a therapeutically effective amount of a thromboxane inhibitor and a pharmaceutically acceptable carrier. The active ingredients together with the inert pharmaceutical excipients are made into pharmaceutical unit dosage form such as tablets and capsules using conventioal pharmacy techniques. The combination can also be administered in separate dosage forms, each having one of the active agents. Such separate unit dosage forms may be packaged together into a pharmaceutical product such as blister packs, which are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

If administered in separate dosage forms, the separate dosage forms are administered such that the beneficial effect of each active agent is realized by the patient at substantially the same time.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

### EXAMPLE 1

### Tablet Preparation

Tablets containing 25.0, 50.0, and 100.0 mg, respectively, of a thromboxane inhibitor are prepared as illustrated below:

TABLE FOR DOSES CONTAINING
FROM 25-100 MG OF THROMBOXANE INHIBITOR

| | Amount-mg | | |
|---|---|---|---|
| thromboxane inhibitor | 25.0 | 50.0 | 100.0 |
| Microcrystalline cellulose | 37.25 | 100.0 | 200.0 |
| Modified food corn starch | 37.25 | 4.25 | 8.5 |
| Magnesium stearate | 0.50 | 0.75 | 1.5 |

All of the active compound, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of active ingredient per tablet.

### EXAMPLE 2

### Wet Granulated Tablet Composition

Tablet dose strengths of between 5 and 125 mg can be accommodated by varying total tablet weight, and the ratio of the first three ingredients. Generally it is preferable to maintain a 1:1 ratio for microcrystalline cellulose : lactose monohydrate.

| Ingredient | Amount/tablet | | | | | | | |
|---|---|---|---|---|---|---|---|---|
| COX-2 Selective | 25 | mg | 12.5 | mg | 10 | mg | 5 | mg |
| Inhibitor | | | | | | | | |
| Microcrystalline | 79.7 | mg | 86 | mg | 87.2 | mg | 89.7 | mg |
| cellulose | | | | | | | | |
| Lactose monohydrate | 79.7 | mg | 86 | mg | 87.2 | mg | 89.7 | mg |
| Hydroxypropyl cellulose | 6 | mg | 6 | mg | 6 | mg | 6 | mg |
| Croscarmellose sodium | 8 | mg | 8 | mg | 8 | mg | 8 | mg |
| Iron oxide | 0.6 | mg | 0.6 | mg | 0.6 | mg | 0.6 | mg |
| Magnesium stearate | 1 | mg | 1 | mg | 1 | mg | 1 | mg |

### EXAMPLE 3

### Directly Compressed Tablet Composition

Tablet dose strengths of between 5 and 125 mg can be accommodated by varying total tablet weight, and the ratio of the first three ingredients. Generally it is preferable to maintain a 1:1 ratio for microcrystalline cellulose: lactose monohydrate.

| Ingredient | Amount per tablet | | | | | | | |
|---|---|---|---|---|---|---|---|---|
| COX-2 Selective | 5 | mg | 10 | mg | 12.5 | mg | 25 | mg |
| Inhibitor | | | | | | | | |
| Microcrystalline | 45 | mg | 42.5 | mg | 113.2 | mg | 106.9 | mg |
| cellulose | | | | | | | | |
| Lactose anhydrate | 45 | mg | 42.5 | mg | 113.2 | mg | 106.9 | mg |
| Croscarmellose sodium | 4 | mg | 4 | mg | 7.5 | mg | 7.5 | mg |
| Magnesium stearate | 1 | mg | 1 | mg | 3.7 | mg | 3.7 | mg |

### EXAMPLE 4

### Hard Gelatin Capsule Composition

Capsule dose strengths of between 1 and 50 mg can be accommodated by varying total fill weight, and the ratio of the first three ingredients. Generally it is preferable to maintain a 1:1 ratio for microcrystalline cellulose : lactose monohydrate.

| Ingredient | Amount per capsule | |
|---|---|---|
| COX-2 Selective Inhibitor | 25 | mg |
| Microcrystalline cellulose | 37 | mg |
| Lactose anhydrate | 37 | mg |
| Magnesium stearate | 1 | mg |
| Hard gelatin capsule | 1 | capsule |

### EXAMPLE 5

### Oral Solution

Solution dose strengths of between 1 and 50 mg/5 mL can be accommodated by varying the ratio of the two ingredients.

| Ingredient | Amount per 5 mL dose |
|---|---|
| COX-2 Inhibitor | 50 mg |
| | to 5 mL with Polyethylene oxide 400 |

### EXAMPLE 6

### Oral Suspension

Suspension dose strengths of between 1 and 50 mg/5 ml can be accommodated by varying the ratio of the first two ingredients.

| Ingredient | Amount per 5 mL dose | |
|---|---|---|
| COX-2 Selective Inhibitor | 101 | mg |
| Polyvinylpyrrolidone | 150 | mg |
| Poly oxyethylene sorbitan monolaurate | 2.5 | mg |
| Benzoic acid | 10 | mg |

to 5 mL with sorbitol
solution (70%)

### EXAMPLE 7

### Combination Tablet Preparation

Tablets containing 25.0, 50.0, and 100.0 mg, respectively, of a thromboxane inhibitor and 25 mg COX-2 selective inhibitor are prepared as illustrated below:

| | Amount-mg | | |
|---|---|---|---|
| Thromboxane Inhibitor | 25.0 | 50.0 | 100.0 |
| COX-2 Selective Inhibitor | 25.0 | 25.0 | 25.0 |
| Microcrystalline cellulose | 37.25 | 100.0 | 175.0 |
| Modified food corn starch | 37.25 | 4.25 | 8.5 |
| Magnesium stearate | 0.50 | 0.75 | 1.5 |

Both active compounds, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of thromboxane inhibitor per tablet, and 25 mg COX-2 selective inhibitor, per tablet.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for the treatment or prophylaxis of COX-2-mediated conditions in patients who are at risk of developing thromboembolic events which comprises administering to said patiens a therapeutically or prophylactically effective amount of a COX-2 selective inhibitor and a cardiovascular protective amount of a thromboxane inhibitor.

2. A method of claim 1 wherein said COX-2 selective inhibitor and said thromboxane inhibitor are adiminstered orally.

3. A method of claim 1 wherein said COX-2 mediated condition is osteoarthritis.

4. A method of claim 1 wherein said COX-2 mediated condition is rheumatoid arthritis.

5. A method of claim 1 wherein said COX-2 mediated condition is selected from Alzeheimer's disease.

6. A method of claim 1 wherein said COX-2 mediated condition is transformation of colonic adenoma into colonic adenocarcinoma.

7. A method of claim 1 wherein said COX-2 selective inhibitor is selected from:
3-phenyl-4-(4-(methylsulfonyl)phenyl)-2-(5H)-furanone;
3-(3,4-difluorophenyl)-4-(4-(methylsulfonyl)phenyl)-2-(5H)-furanone;
5,5-dimethyl-4-(4-(methylsulfonyl)phenyl-3-(3-fluorophenyl)-5H-furan-2-one;
5,5-dimethyl-4(4-(methylsulfonyl)phenyl)-3-(2-propoxy)-5H-furan-2-one;
5-chloro-3(4-(methylsulfonyl)phenyl)-2-(2-methyl-5pyridinyl)pyridine;
2-(3,5-difluorophenyl)-3-(4-(methylsulfonyl)phenyl)-2-cyclopenten-1-one;
5(S)-5-ethyl-5-methyl-4-(4-(methylsulfonyl)phenyl)-3-(2-propoxy)-5H-furan-2-one;
5-ethyl-5-methyl-4-(4-(methylsulfonyl)phenyl)-3-(3,4-difluorophenyl)-5H-furan-2-one;
3-((2-thiazolyl)methoxy)-4-(4-(methylsulfonyl)phenyl)-5,5-dimethyl-5H-furan-2-one;
3-propyloxy-4-(4-(methylsulfonyl)phenyl)-5,5-dimethyl-5H-furan-2-one;
3-(1-cyclopropylethoxy)-5,5-dimethyl-4-(4-methylsulfonyl)phenyl)-5H-furan-2-one;
sodium 2-(4-chlorophenyl)-3-(4-(methylsulfonyl)phenyl)-4-oxo-2-pentenoate;
3-(cyclopropylmethoxy)-5,5-dimethyl-4-(4-(methylsulfonyl)phenyl)-5H-furan-2-one;
3-(cyclopropylmethoxy)-5,5-dimethyl-4-(4-(methylsulfonyl)phenyl)-2,5-dihydrofuran-2-ol;
3-isopropoxy-5,5-dimethyl-4-(4-(methylsulfonyl)phenyl)-2,5-dihydrofuran-2-ol;
5,5-dimethyl-3-(3-fluorophenyl)-2-hydroxy-4-(4-(methylsulfonyl)phenyl)-2,5-dihydrofuran and
5-Chloro-3-(4-(methylsulfonyl)phenyl)-2-(3-pyridinyl)pyridine.

8. A method of claim 1 wherein said COX-2 selective inhibitor is 3-phenyl-4-(4-(methylsulfonyl)phenyl)-2-(5H)-furanone or 5-chloro-3-(4-(methylsulfonyl)phenyl)-2-(2-methyl-5-pyridinyl)pyridine.

9. A method of claim 1 wherein said COX-2 selective inhibitor is 4-[5-(4-methylphenyl)-3-(trifluoromethyl)-1H-pyrazol-1-yl]benzenesulfonamide or 4-(5-methyl-3-phenylisoxazol-4-yl)benzenesulfonamide.

10. A method of claim 1 whrein said thromboxane inhibitor is a thromboxane synthase inhibitor.

11. A method of claim 1 wherein said thromboxane inhibitor is a thromboxane receptor antagonists.

12. A pharmaceutical composition comprising a therapeutically or prophylactically effective amount of a COX-2 selective inhibitor and a cardiovascular protective amount of a thromboxane inhibitor, and a pharmaceutically acceptable carrier in a unit dosage form.

13. A pharamceutical composition of claim 12 wherein said COX-2 selective inhibitor is selected from the group consisting of:
3-phenyl-4-(4-(methylsulfonyl)phenyl)-2-(5H)-furanone;
3-(3,4-difluorophenyl)-4-(4-(methylsulfonyl)phenyl)-2-(5H)-furanone;
5,5-dimethyl-4-(4-(methylsulfonyl)phenyl)-3-(3-fluorophenyl)-5H-furan-2-one;
5,5-dimethyl-4-(4-(methylsulfonyl)phenyl)-3-(2-propoxy)-5H-furan-2-one;
5-chloro-3-(4-(methylsulfonyl)phenyl)-2-(2-methyl-5-pyridinyl)pyridine;
2-(3,5-difluorophenyl)-3-(4-(methylsulfonyl)phenyl)-2-cyclopenten-1-one;
5(S)-5-ethyl-5-methyl-4-(4-(methylsulfonyl)phenyl)-3-(2-propoxy)-5H-furan-2-one;
5-ethyl-5-methyl-4-(4-(methylsulfonyl)phenyl)-3-(3,4-difluorophenyl)-5H-furan-2-one;
3-((2-thiazolyl)methoxy)-4-(4-(methylsulfonyl)phenyl)-5,5-dimethyl-5H-furan-2-one;
3-propyloxy-4-(4-(methylsulfonyl)phenyl)-5,5-dimethyl-5H-furan-2-one;
3-(1-cyclopropylethoxy)-5,5-dimethyl-4-(4-methylsulfonyl)phenyl)-5H-furan-2-one;
sodium 2-(4-chlorophenyl)-3-(4-(methylsulfonyl)phenyl)-4-oxo-2-pentenoate;
3-(cyclopropylmethoxy)-5,5-dimethyl-4-(4-(methylsulfonyl)phenyl)-5H-furan-2-one;
3-(cyclopropylmethoxy)-5,5-dimethyl-4-(4-(methylsulfonyl)phenyl)-2,5-dihydrofuran-2-ol;
3-isopropoxy-5,5-dimethyl-4-(4-(methylsulfonyl)phenyl)-2,5-dihydrofuran-2-ol;
5,5-dimethyl-3-(3-fluorophenyl)-2-hydroxy-4-(4-(methylsulfonyl)phenyl)-2,5-dihydrofuran and
5-Chloro-3-(4-(methylsulfonyl)phenyl)-2-(3-pyridinyl)pyridine.

14. A pharmaceutical composition of claim 12 wherein said COX-2 selective inhibitor is 3-phenyl-4-(4-(methylsulfonyl)phenyl)-2-(5H)-furanone or 5-chloro-3-(4-(methylsulfonyl)phenyl)-2-(2-methyl-5-pyridinyl)pyridine.

15. A pharmaceutical composition of claim 12 wherein said COX-2 selective inhibitor is 4-[5-(4-methylphenyl)-3-(trifluoromethyl)-1H-pyrazol-1-yl]-benzenesulfonamide or 4-(5-methyl-3-phenylisoxazol-4-yl)benzenesulfonamide.

16. A pharmaceutical product comprising (1) a therapeutically or prophylactically effective amount of a COX-2 selective inhibitor in a first oral unit dosage form, (2) a cardioprotective amount of a thromboxane inhibitor in a second oral unit dosage form, and (3) instructions for concurrent or sequential administration of said pharmaceutical product to a patient in need thereof.
